(19) United States
(12) Patent Application Publication
(10) Pub. No.: US 2022/0339209 A1
(43) Pub. Date: Oct. 27, 2022

(54) ISOLATION, IDENTIFICATION AND APPLICATION OF STREPTOCOCCUS SP. 121

(71) Applicants: Lina NIU, HAIKOU CITY (CN); Peipei Cao, HAIKOU CITY (CN); Qiang Wu, HAIKOU CITY (CN); Shoukui Hu, HAIKOU CITY (CN); Zhiling Chen, HAIKOU CITY (CN); Bin Qiao, HAIKOU CITY (CN); Feifei Yin, HAIKOU CITY (CN); Xiuji Cui, HAIKOU CITY (CN)

(72) Inventors: Lina NIU, HAIKOU CITY (CN); Peipei Cao, HAIKOU CITY (CN); Qiang Wu, HAIKOU CITY (CN); Shoukui Hu, HAIKOU CITY (CN); Zhiling Chen, HAIKOU CITY (CN); Bin Qiao, HAIKOU CITY (CN); Feifei Yin, HAIKOU CITY (CN); Xiuji Cui, HAIKOU CITY (CN)

(21) Appl. No.: 17/725,462

(22) Filed: Apr. 20, 2022

(30) Foreign Application Priority Data
Apr. 21, 2021 (CN) 2021104328937

(51) Int. Cl. A61K 35/74; C12N 1/20; C12N 1/02

(52) U.S. Cl. CPC A61K 35/74; C12N 1/205; C12N 1/02; C12R 2001/46

(57) ABSTRACT

The present invention provides a Streptococcus, which is a new species of the genus of Streptococcus, named as Streptococcus sp. 121, and is registered and deposited in GuangDong Microbial Culture Collection Center with the accession number of GDMCC No: 61195. After fermentation culture, the Streptococcus sp. 121 of the present invention can produce a stronger bacteriostatic active substance, which has relatively obvious antimicrobial effects on Klebsiella pneumoniae, Shigella dysenteriae, and the like, has a broad development space in prevention and treatment of diseases caused by Klebsiella pneumoniae and Shigella dysenteriae, and has a very good development and application prospect in antimicrobial drugs.

### TECHNICAL FIELD

The present invention belongs to the field of microorganisms, and in particular relates to isolation, identification and application of Streptococcus sp. 121.

### BACKGROUND

Streptococcus is a type genus belongs to the family Streptococcaceae, within the order Lactobacillales , in the class Bacilli of the phylum Firmicutes. This genus of bacteria is a kind of common bacteria of pyogenic cocci, which widely exist in nature, human and animal feces and healthy people's nasopharynx, and most of them are not pathogenic. Medically important Streptococcus mainly include Streptococcus pyogenes, Streptococcus viridans, Streptococcus pneumoniae, Streptococcus agalactiae and the like. With the continuous progress and development of bacterial isolation and identification methods, especially the wide application of molecular classification and identification methods, more and more Streptococcus have been found. Meanwhile, up to 2020, this genus has included 160 species and 29 subspecies (http://www.bacterio.net/streptococcus.htmL). In recent years, there have been many research reports on new species of Streptococcus from animal sources, especially from wild animal sources.

In recent years, with the development of taxonomical study in molecular level, especially the application of 16S rRNA sequence homology and chemical classification, the classification of the genus of Streptococcus has changed greatly. In 1995, Kawamura first divided the genus of Streptococcus into 6 species groups, i.e., a Pyogenic group, a Mitis group, an Anginosus group, a Mutans group, a Salivarius group and a Bovis group, respectively, based on the study of molecular systematics of 16S rRNA sequence. Among them, the bacteria in the Mitis group include Streptococcus mitis, Streptococcus sanguis (with 3 biotypes), Streptococcus parasanguis, Streptococcus gordonii, Streptococcus crista, Streptococcus oralis and Streptococcus pneumoniae. The bacteria included in the Anginosus group include Streptococcus anginosus, Streptococcus constellatus and Streptococcus intermadius. The Mutans group includes Streptococcus mutans, Streptococcus sobrinus, Streptococcus rattus, Streptococcus muris-ratti, Streptococcus downei and Streptococcus macacae, etc. The Salivarius group includes Streptococcus sahvarius, Streptococcus vestibularis and Streptococcus thermophilus. The Bovis group includes Streptococcus bovis, Streptococcus alactolyticus and Streptococcus equi. However, S. acidominimus which can produce α-hemolysis, and S. suis (which is presented as β hemolysis on a horse blood medium), have not yet been grouped, while S. ferus isolated from a rat may be a candidate strain included in the Mutans group, which needs to be clearly classified. Then, some scholars used other gene sequences other than the 16S rRNA sequence, such as groEL and gyrB, to evaluate the phylogenetic relationship within the genus of Streptococcus, and further revised the grouping of genus of Streptococcus. The Streptococcus anginosus, Streptococcus constellatus and Streptococcus intermadius originally included in the Anginosus group, were classified into a Mitis group A. S. ferus was classified into the Mutans group. S. acidominimus and S. suis were classified into a suis group. Up to now, the accepted grouping of the genus of Streptococcus includes a pyogenes group, the Mitis group (subdivided into group A and group B), the Mutans group, the Salivarius group, the Bovis group, and the suis group. In the article “Molecular phylogeny and a taxonomic proposal for the genus Streptococcus” published in 2015, Pontigl proposed to establish a new group of S. gordonii, S. pluranimahum and S. sobrinus on the grouped basis.

An antimicrobial drug generally refers to a drug with bactericidal or bacteriostatic activities, including various antibiotics, sulfonamides, imidazoles, nitroimidazoles, quinolones and the like chemical synthetic drugs. It also refers to certain products obtained by culturing microorganisms such as bacteria, actinomycetes and fungi, or the same or similar substances produced by a chemical semi-synthesis method. The antimicrobial drug is a kind of drug that is widely used and important in clinic. With the long-term use of the antimicrobial drug, drug resistance against the antimicrobial drug is constantly produced in environmental bacteria, so it is required to develop a novel antimicrobial drug to deal with this situation.

Currently, most of the antimicrobial compounds used clinically come from primary and secondary metabolites produced by terrestrial soil and marine microorganisms. They are of various kinds and have different functions, and have made considerable contributions to the development of health undertaking in human society. However, with people's long-term research and development, obtaining novel antimicrobial active substances has gradually become a major problem existed in microbial screening.

In recent years, the research reports on the diversity and biological functions of intestinal microorganisms in murines have gradually increased. Intestinal microorganisms are abundant in species and large in quantity, which play an important role in nutrition metabolism, growth and development, reproduction and multiplication, immune defense, and the like aspects of a host. Murines, as the main group of wild animals, can live in a dark and humid environment with a large number of pathogenic bacteria for a long time, and they have tenacious vitality, strong reproduction, stress and disease resistances, and the like capabilities, suggesting that this may be related to the abundant microbial flora in their intestines that can resist the infection with foreign microorganisms (including pathogenic bacteria). Papers published in Cell show that the intestinal microflora of wild murines promotes health of a host and improve disease resistance, which indicates that the capability of wild murines to adapt to the external environment and defend against the invasion of foreign pathogenic bacteria may be closely related to the antimicrobial activity of intestinal Streptococcus.

In this study, Streptococcus sp. 121, a new unknown strain of Streptococcus, was isolated from an intestinal sample of wild Rattus norvegicus in the central mountainous area of Hainan Island. This bacterium can produce natural substances with antimicrobial activities and has a wide antimicrobial spectrum, which plays an important role in the process of Rattus norvegicus resisting the invasion of foreign pathogenic bacteria. The study of this bacterium is helpful to provide guidance for acquiring Streptococcus resources of new natural active substances from the intestinal tract of rodents in a tropical environment.

### SUMMARY

An objective of the present invention is to overcome the shortcomings in the prior art, by providing a Streptococcus sp. 121, which has relatively strong bacteriostatic activity and a good development and application prospect in antimicrobial drugs.

A first aspect of the present invention provides a Streptococcus, which is a new species of the genus of Streptococcus, named as Streptococcus sp. 121, and is registered and deposited in GuangDong Microbial Culture Collection Center with the accession number of GDMCC No: 61195.

A second aspect of the present invention provides application of the Streptococcus according to the first aspect of the present invention in antagonizing Klebsiella pneumoniae.

A third aspect of the present invention provides application of the Streptococcus according to the first aspect of the present invention in preparation of a medicament for preventing and treating a condition caused by Klebsiella pneumoniae.

A fourth aspect of the present invention provides application of the Streptococcus according to the first aspect of the present invention in antagonizing Shigella dysenteriae.

A fifth aspect of the present invention provides application of the Streptococcus according to the first aspect of the present invention in preparation of a medicament for preventing and treating a condition caused by Shigella dysenteriae.

A sixth aspect of the present invention provides a fermentation broth of the Streptococcus according to the first aspect of the present invention, or a supernatant fermentation broth obtained after centrifugation of the fermentation broth, or a extract of the supernatant fermentation broth.

A seventh aspect of the present invention provides application of the fermentation broth of Streptococcus, or the supernatant fermentation broth obtained after centrifugation of the fermentation broth, or the extract of the supernatant fermentation broth according to the sixth aspect of the present invention in antagonizing Klebsiella pneumoniae.

A eighth aspect of the present invention provides application of the fermentation broth of Streptococcus, or the supernatant fermentation broth obtained after centrifugation of the fermentation broth, or the extract of the supernatant fermentation broth according to the sixth aspect of the present invention in preparation of a medicament for preventing and treating a condition caused by Klebsiella pneumoniae.

A ninth aspect of the present invention provides application of the fermentation broth of Streptococcus, or the supernatant fermentation broth obtained after centrifugation of the fermentation broth, or the extract of the supernatant fermentation broth according to the sixth aspect of the present invention in antagonizing Shigella dysenteriae.

A tenth aspect of the present invention provides application of the fermentation broth of Streptococcus, or the supernatant fermentation broth obtained after centrifugation of the fermentation broth, or the extract of the supernatant fermentation broth according to the sixth aspect of the present invention in the preparation of a medicament for preventing and treating a disease caused by Shigella dysenteriae.

The Streptococcus sp. 121 of the present invention is Gram-positive, Catalase-negative, oval and arranged in a chainlike form. It is determined for the 16S rRNA sequence by PCR technology, and subjected to alignment with the nucleotide sequence in Genbank by Blast. The results showed that Streptococcus sp. 121 has the highest similarity in the 16S rRNA sequence with Streptococcus danieliae, Streptococcus sanguinis, Streptococcus koreensis and Streptococcus cristatus, with identity values of 97.72%, 97.14%, 96.75% and 96.68%, respectively. Auxiliary identification is further conducted by utilizing the conserved gene gyrB, in which its amplified sequence is subjected to Blast with the sequence in the Genbank. The results show that gyrB gene of Streptococcus sp. 121 has the highest similarity with that of Streptococcus peroris (77.40%). By conducting Pacbio whole genome sequencing analysis of Streptococcus sp. 121, a DNA-DNA molecular hybridization experiment has showed that the hybridization ratio between Streptococcus sp. 121 and other known Streptococcus ranges from 21.4%-29.3%, which are all lower than a threshold of 70%. Studies on its phenotypic characteristics and phylogeny have showed that Streptococcus sp. 121 is a new species of the genus of Streptococcus, with a genome size of 2,011,914 bp, containing 1,967 genes and a GC content of 42.5 mol %.

After fermentation culture, the Streptococcus sp. 121 of the present invention can produce a stronger bacteriostatic active substance, which has relatively obvious antimicrobial effects on Klebsiella pneumoniae, Shigella dysenteriae, and the like, has a broad development space in prevention and treatment of diseases caused by Klebsiella pneumoniae and Shigella dysenteriae, and has a very good development and application prospect in antimicrobial drugs.

### BRIEF DESCRIPTION OF DRAWINGS

FIG. 1 is a diagram showing the colony morphology of Streptococcus sp. 121 in a brain heart infusion (BHI) sheep blood agar plate.

FIG. 2 is a diagram showing the Gram staining of Streptococcus sp. 121.

FIG. 3 is a perspective electron microscope graph of Streptococcus sp. 121.

FIG. 4 is a diagram showing the phylogenetic relationship between the Streptococcus sp. 121 constructed based on the 16S rRNA gene sequence and a type strain in the main groups of the genus of Streptococcus.

FIG. 5 shows the phylogenetic tree of Streptococcus sp. 121 based on a conserved gene sequence.

FIG. 6 is a diagram showing the result of a bacteriostatic test of the fermentation broth of Streptococcus sp. 121 against Klebsiella pneumoniae.

FIG. 7 is a diagram showing the result of a bacteriostatic test of the fermentation broth of Streptococcus sp. 121 against Shigella dysenteriae.

### DETAILED DESCRIPTION

The present invention will be further described below with reference to specific examples for better understanding of the present invention.

The present invention provides a Streptococcus, which is a new species of the genus of Streptococcus, named as Streptococcus sp. 121, and is registered and deposited in GuangDong Microbial Culture Collection Center on Sep. 16, 2020 with the accession number of GDMCC No: 61195. The Streptococcus sp. 121 of the present invention is isolated and screened from intestinal samples of Rattus norvegicus in the central mountainous area of Hainan Island.

### 1. Morphological Observation Of New Streptococcus Species

### 1.1 Source Of Strains

Intestinal specimens of Rattus norvegicus in Hainan Island. The intestinal mucosa specimens of healthy adult Rattus norvegicus were taken, and tissues of intestinal segments of Rattus norvegicus were cut by aseptic operations and put into a grinder for grinding (about 20 mg). The ground mucosa homogenate was diluted with 300 μL of brain heart infusion (BHI), then transferred into a 2 mL sterile centrifuge tube containing 1.5 mL of PBS for high-speed vortex, and then centrifuged at a low speed. About 200 microliters of the supernatant was pipetted, evenly coated on a BHI sheep blood plate with a spreading rod, and placed and incubated in a 5% CO₂ incubator at 37° C. The aforementioned operations were conducted in triplicate.

The formulation method of the brain heart infusion (BHI) sheep blood agar plate: 4.0 g of bovine brain infusion powder, 4.0 g of bovine heart infusion powder , 5.0 g of peptone, 16.0 g of casein peptone, 5.0 g of sodium chloride, 2.0 g of glucose, 2.5 g of disodium hydrogen phosphate, 13.5 g of agar, were added with water to adjust the total volume to 1,000 mL. The mixture was subjected to autoclaving at 121° C. for 15 minutes, cooled to about 50° C., slowly added with 80 mL of defibered sheep blood, and poured into a plate to formulate a 8% BHI sheep blood agar plate.

### 1.2 Morphological Observation

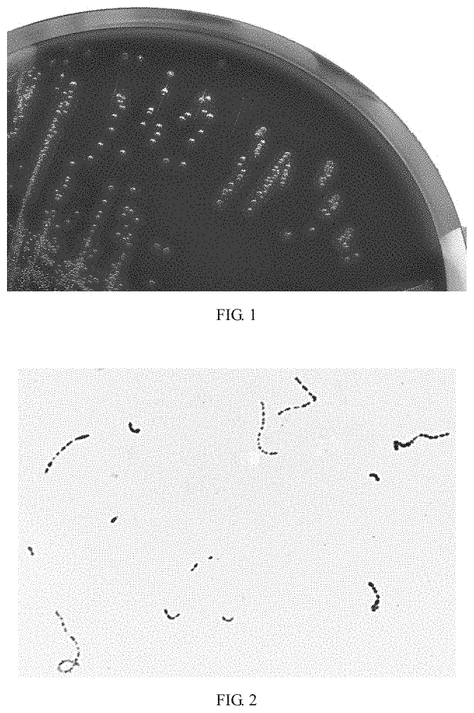

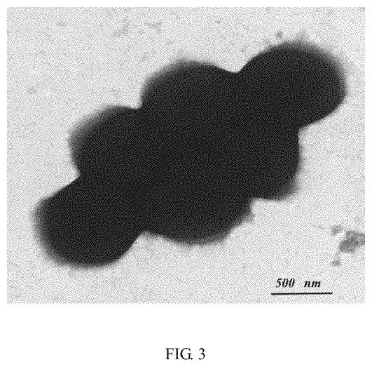

The isolated and purified new Streptococcus sp. was transferred into the BHI sheep blood agar plate, and placed and incubated in a 5% CO₂ incubator at 37° C. for 24-48 h. A tiny needle-like colony with a diameter of 0.6-1.0 mm, which was off-white, rounded and raised, and moist and has neat edges (FIG. 1), was formed after Streptococcus sp. 121 had been incubated on the BHI sheep blood agar medium at 37° C. for 24 h. After continuous culture of it for 48 h, a grassy hemolytic ring of about 1 mm appeared around its colony. When observed under an optical microscope, the strain was Gram-positive, and individual bacteria were spherical and arranged in pairs or in a form of several short chains (FIG. 2). When observed under an electron microscope, the bacteria showed the typical characteristics of Streptococcus: individual bacteria exhibited a spherical or oval shape with a diameter of about 0.5-1.0 and the bacteria were arranged in pairs or in a form of several short chains, without spores and flagella (FIG. 3).

### 1.3 Sodium Chloride Tolerance Test Of Streptococcus Sp. 121

TABLE 1
Result of sodium chloride tolerance test of Streptococcus sp. 121

| | 1.5 g/L | 2.5 g/L | 3.5 g/L | 6.5 g/L |
|---|---|---|---|---|
| Streptococcus | + | + | − | − |
| sp. 121 | | | | |

Streptococcus sp. 121 grew on a BHI medium containing 2.5% NaCl, but did not grow on a BHI medium containing more than 2.5% NaCl.

### 1.4 Temperature Tolerance Test Of Streptococcus Sp. 121

TABLE 2
Result of temperature tolerance test of Streptococcus sp. 121

| | 4° C. | 15° C. | 22° C. | 35° C. | 37° C. | 42° C. |
|---|---|---|---|---|---|---|
| Streptococcus | — | + | + | + + + | + + + + | + + |
| sp. 121 | | (after | | | | |
| | | 48 h) | | | | |

Streptococcus sp. 121 grew when incubated at 22, 35, 37 and 42° C., but did not grow when incubated at 4 and 15° C.

It could be preliminarily determined that Streptococcus sp. 121 belongs to the genus of Streptococcus by identification from the morphological aspect. However, only from the colony morphology and microscopic observation, it is not clear which species of the genus of Streptococcus they belong to. In order to further determine its taxonomic status, it is necessary to carry out biochemical and molecular biological analysis on it.

### 2. Identification By Biochemical Experiment

### 2.1 Identification By API Rapid ID 32 Strep Biochemical Reaction

The purified bacteria were inoculated into the BHI sheep blood agar plate, and placed and incubated in a CO₂ incubator at 37° C. for 18-24 h. Firstly, the hemolysis type (α or β) and chromogenic condition (as a supplementary experiment) were recorded. Then, enough colonies were picked into an ampoule bottle containing 2 mL API suspension medium by a cotton swab or inoculating loop, and determined by a turbidimeter to obtain a bacteria suspension with a McFarland turbidity of 4.0. Then, the bacteria suspension was subpackaged into each reaction well with 55 μL per well, gently shaken and evenly mixed, covered with a reaction cover, and placed and incubated in a CO₂ incubator at 37° C. for 4-4.5 h. Then the result was observed, as shown in Table 3.

### 2.2 Identification By API 50 CHB Biochemical Reaction

The purified bacteria were inoculated into the BHI sheep blood agar plate, and placed and incubated in a CO₂ incubator at 37° C. for 18-24 h. Then, enough colonies were picked into 1 mL distilled water by a cotton swab or inoculating loop, and determined by a turbidimeter to obtain a bacteria suspension with a McFarland turbidity of 4.0. Then, the bacteria suspension was transferred into an ampoule bottle containing a API 50 CHB medium, evenly mixed, then subpackaged into each reaction well with 100 μL per well, finally added dropwise with glycerol per well for moisture retention, covered with a reaction cover, and placed and incubated in a CO₂ incubator at 37° C. for 24 h. Then the result was observed, as shown in Table 3.

### 2.3 Identification By API ZYM Biochemical Reaction

The purified bacteria were inoculated into the BHI sheep blood agar plate, and placed and incubated in a CO₂ incubator at 37° C. for 18-24 h. Then, enough colonies were picked into a ampoule bottle containing 2 mL API suspension medium by a cotton swab or inoculating loop, and prepared into a bacteria suspension with a McFarland turbidity of 6.0. Then, the bacteria suspension was subpackaged into each reaction well with 65 μL per well, gently shaken and evenly mixed, covered with a reaction cover, and placed and incubated in a CO₂ incubator at 37° C. for 4-4.5 h. Then the result was observed, as shown in Table 3.

TABLE 3
Comparison of biochemical reaction results between Streptococcus
sp. 121 and other closely related Streptococcus

| | Streptococcus | Streptococcus | Streptococcus |
|---|---|---|---|
| Reaction Name | sp.121 | danieliae | sanguinis |
| β-methyl-D-xyloside | − | − | + |
| Galactose | − | − | + |
| Mannose | + | − | + |
| Arbutin | + | + | − |
| Esculin | + | − | − |
| Salicin | + | − | − |
| Cellobiose | + | − | − |
| Lactose | + | − | + |
| Inulin | − | − | + |
| Alkaline phosphatase | − | + | − |
| β-glucosidase | − | − | + |
| β-galactosidase | + | − | − |
| Arginine dihydrolase | − | − | + |

### 3. Classification And Identification Of 16S rRNA Of New Streptococcus Sp.

A Single colony of suspected new species (preliminarily judged based on size, morphology and color of the colony) was picked by a sterile Tip head or a sterilized toothpick to serve as a PCR reaction template, and PCR amplification of the colony was carried out by using primers 27F-1492R (27F: 5′-AGAGTTTGATCMTGGCTCAG-3′, and 1492R: 5′-GGYTACCTTGTTACGACTT-3′). The amplified products were sent to Guangzhou Tianyi Huiyuan Biotechnology Co., Ltd. for sequencing.

The sequencing result was put into GenBank, and subjected to alignment with known nucleic acid sequences in the GenBank by BLAST. The result had showed that the identity value between Streptococcus sp. 121 and each of all the nucleic acid sequences was lower than 98%. The similarity in 16S rRNA sequence of Streptococcus danieliae, Streptococcus sanguinis, Streptococcus koreensis and Streptococcus cristatus was the highest, with identity values of 97.72%, 97.14%, 96.75% and 96.68% respectively. According to the principle that identities ≥94.5% were determined of the same genus and identities ≥98.7% were determined of the same species, Streptococcus sp. 121 was preliminarily determined as a suspected new species of the genus of Streptococcus.

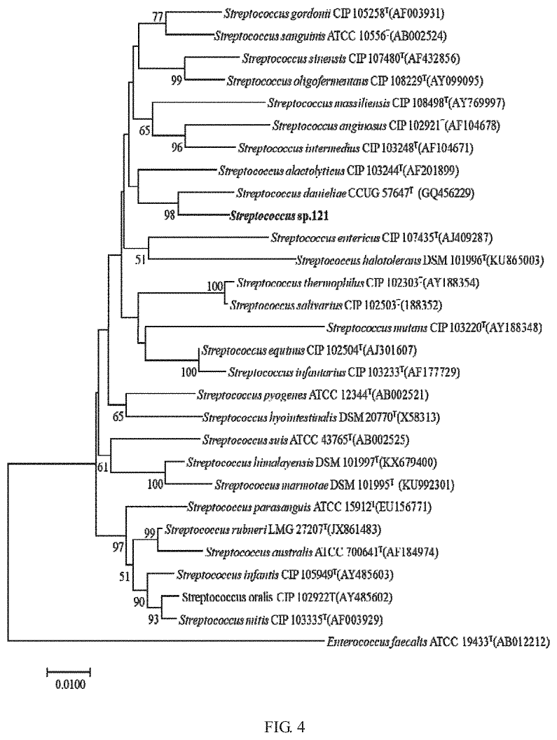

The resultant sequences were spliced by employing SeqMan software and DNA star software, and determined in forward and reverse directions, and the vector and primer sequences were removed therefrom. Thereafter, a phylogenetic tree is constructed by using MAGA 7.0 software and employing a proximity-connection method and a maximum likelihood method, and finally the generated phylogenetic tree was verified by Bootstrap. Enterococcus faecalis ATCC 19433^{T} of the genus of Enterococcus was used as an external group. The result was as shown in FIG. 4, and it could be seen that Streptococcus sp. 121 was located in an independent evolutionary branch on a clustering tree and clustered with Streptococcus danieliae, and its Bootstrap support rate was 98%, showing a close genetic relationship. In alignment by Blast, the identity value of Streptococcus sp. 121 with each of other known species of the genus of Streptococcus was lower than 98%, showing a far genetic relationship. The research had shown that Streptococcus sp. 121 belonged to a new species of the genus of Streptococcus, as supported from the classification level of 16S rRNA.

### 4. Classification And Identification Of New Streptococcus Species By Conserved Genes

In order to further identify the taxonomic status of the new Streptococcus species and study their genetic relationship with known Streptococcus, this topic carried out taxonomic identification research on its conservative gene gyrB.

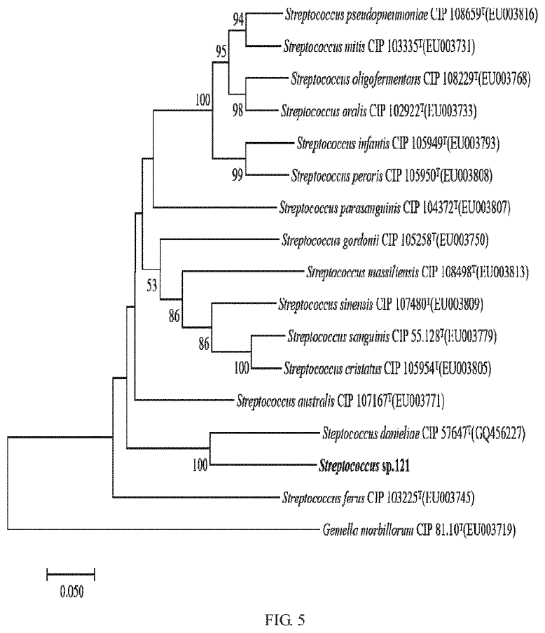

Blast alignment analysis was conducted between a gyrB amplified sequence of Streptococcus sp. 121 and the sequence in the Genbank, and the result had shown that all of closely related species with high identity values to it belonged to the “Mitis group” of the genus of Streptococcus. It could be seen from FIG. 5 that Streptococcus sp. 121, as a single branch, was clustered with Streptococcus danieliae, and its Bootstrap support rate was 100%, showing a close genetic relationship (see FIG. 5), which indicated that Streptococcus sp. 121 belonged to a new species in the genus of Streptococcus as supported from the classification level of the conserved gene gyrB, and this bacterium had a close genetic relationship with Streptococcus danieliae. The result was consistent with the phylogenetic tree result constructed from its 16S rRNA sequence.

### 5. Genomic analysis of new Streptococcus species

In this topic, after the pure culture of Streptococcus sp. 121 was obtained, the whole genome sequencing was conducted, predictive parsing of the basic characteristics of its genome was conducted, and the genetic evolution analysis of the type strain and other known species of the genus of Streptococcus was conducted.

### 5.1 Whole Genome Sequencing And Assembly

The whole genome sequencing work of Streptococcus sp. 121 was completed by Beijing Novogene Co., Ltd. The whole genome of Streptococcus sp. 121 was determined by a single molecule real-time sequencing technology of Pacific Biosciences, a third-generation sequencing platform. The sequencing data were filtered by SMRT Analysis 2.3.0 software package for quality control, and a complete circular genomic sequence was spliced and assembled.

### 5.2 Analysis Of Basic Characteristics Of Genome

GeneMarkS (http://topaz.gatech.edu/) software was used for CDS predicting of coding gene, and tRNAscan-SE and rRNAmmer software were used for predicting tRNAs and rRNAs in the genome. IslandPath-DIOMB software was utilized to predict the gene island. In this study, the whole genome sequencing of Streptococcus sp. 121 isolated from the intestinal samples of Rattus norvegicus in Hainan Island was conducted by Pacific Biosciences (PacBio), a third-generation sequencing platform. The full length of the genome of Streptococcus sp. 121 was 2,011,914 bp, with a G+C content of 42.5 mol %, encoding 1,967 genes.

### 6. Genomic DNA-DNA Hybridization

On-line genomic hybridization was conducted between the whole genome sequence of Streptococcus sp. 121 and representative sequences of different species of the genus of Streptococcus that could be searched in the current GenBank database by using DNA-DNA hybridization online software (genome and genome distance calculator, GGDC). The DNA-DNA hybridization similarity of more than 70% was divided into the same species, and the results were shown in Table 4.

As could be seen from Table 4, the hybridization ratio between Streptococcus sp. 121 and other known Streptococci was between 21.4%-29.3%, each being lower than the threshold of 70%, indicating that Streptococcus sp. 121 did not belong to any known species, but is a new species of the genus of Streptococcus.

TABLE 4
Result of hybridization between Streptococcus sp. 121 and genomes of different known Streptococcus
species

| | | | | | difference |
|---|---|---|---|---|---|
| | | DDH | | Prob.DDH | in % G + |
| Reference | GenBank accession No. | (%) | Distance | (%) | C |
| S.danieliae | GCA_009767945.1 | 23.4 | 0.1866 | 0 | 1.92 |
| S.acidominimus | NZ_LT906454.1 | 25.7 | 0.169 | 0.01 | 2.58 |
| S.alactolyticus | NZ_VUNP00000000.1 | 24.8 | 0.1759 | 0.01 | 1.99 |
| S.australis | NZ_LS483444.1 | 29.1 | 0.1472 | 0.07 | 0.56 |
| S.azizii | NZ_MSPR00000000.1 | 24.5 | 0.1777 | 0.01 | 0.26 |
| S.bovimastitidis | NZ_LZDD00000000.1 | 22.7 | 0.193 | 0 | 5.16 |
| S.caballi | NZ_ARCA00000000.1 | 24.4 | 0.1789 | 0.01 | 2.13 |
| S.canis | NZ_LR134293.1 | 29.2 | 0.1469 | 0.07 | 2.64 |
| S.castoreus | NZ_AUKZ00000000.1 | 27.5 | 0.1571 | 0.03 | 4.69 |
| S.chosunense | NZ_RBCK00000000.1 | 25 | 0.1738 | 0.01 | 2.35 |
| S.criceti | NZ_UHFB00000000.1 | 26 | 0.1671 | 0.02 | 0.27 |
| S.cristatus | NZ_LS483383.1 | 28 | 0.1537 | 0.04 | 0.1 |
| S.cuniculi | NZ_MSJM00000000.1 | 24.7 | 0.1766 | 0.01 | 0.94 |
| S.defectivus | NZ_ACIN00000000.3 | 21.4 | 0.2048 | 0 | 4.48 |
| S.devriesei | NZ_AUIN00000000.1 | 23.7 | 0.1841 | 0 | 0.7 |
| S.didelphis | NZ_ARCB00000000.1 | 25.2 | 0.1727 | 0.01 | 6.72 |
| S.downei | NZ_UHFA00000000.1 | 27.1 | 0.1594 | 0.03 | 1.2 |
| S.entericus | NZ_ARCC00000000.1 | 24.2 | 0.1807 | 0.01 | 2.13 |
| S.equinus | NZ_UHFK00000000.1 | 28.7 | 0.1496 | 0.06 | 5.01 |
| S.ferus | NZ_LS483343.1 | 24.6 | 0.1774 | 0.01 | 0.34 |
| S.gallolyticus | NZ_CP018822.1 | 26.5 | 0.1633 | 0.02 | 4.8 |
| S.garvieae | NC_015930.1 | 25.6 | 0.1696 | 0.01 | 3.67 |
| S.gwangjuense | NZ_CP032621.1 | 26.9 | 0.161 | 0.02 | 2.3 |
| S.halichoeri | NZ_WLZU00000000.1 | 25.3 | 0.1716 | 0.01 | 0.93 |
| S.halotolerans | NZ_CP014835.1 | 25.3 | 0.1722 | 0.01 | 3.25 |
| S.henryi | NZ_AQYA00000000.1 | 24.7 | 0.1768 | 0.01 | 3.91 |
| S.himalayensis | NZ_CP016953.1 | 27.2 | 0.1591 | 0.03 | 1.23 |
| S.hyointestinalis | NZ_UHFN00000000.1 | 25.9 | 0.1675 | 0.02 | 0.37 |
| S.hyovaginalis | NZ_ATVP00000000.1 | 24.1 | 0.1809 | 0.01 | 2.64 |
| S.ictaluri | NZ_AEUX00000000.2 | 25.4 | 0.1709 | 0.01 | 4.33 |
| S.infantis | NZ_AEVD00000000.1 | 26.3 | 0.1647 | 0.02 | 3.52 |
| S.koreensis | NZ_CP032620.1 | 27.7 | 0.1557 | 0.04 | 0.42 |
| S.lutetiensis | NZ_LS483403.1 | 25.5 | 0.1703 | 0.01 | 4.79 |
| S.macacae | NZ_AEUW00000000.2 | 26.9 | 0.161 | 0.02 | 4.69 |
| S.marimammalium | NZ_ARCD00000000.1 | 23.8 | 0.1833 | 0 | 9.32 |
| S.marmotae | NZ_CP015196.1 | 25.6 | 0.1699 | 0.01 | 1.58 |
| S.massiliensis | NZ_ARCE00000000.1 | 26.3 | 0.165 | 0.02 | 0.98 |
| S.merionis | NZ_LT906439.1 | 25.4 | 0.1709 | 0.01 | 0.75 |
| S.minor | NZ_AQYB00000000.1 | 24.7 | 0.1767 | 0.01 | 1.4 |
| S.mutans | NZ_LS483349.1 | 24.7 | 0.1761 | 0.01 | 5.63 |
| S.oralis | NZ_LR134336.1 | 25.2 | 0.1724 | 0.01 | 0.99 |
| S.orisratti | NZ_ARCG00000000.1 | 24.9 | 0.1751 | 0.01 | 3.97 |
| S.ovis | NZ_ARCH00000000.1 | 25 | 0.1739 | 0.01 | 2.4 |
| S.ovuberis | NZ_JAAXPR000000000.1 | 23.5 | 0.1863 | 0 | 0.26 |
| S.pantholopis | NZ_CP014699.1 | 27.9 | 0.1546 | 0.04 | 0.53 |
| S.parasanguinis | NC_015678.1 | 26.1 | 0.1659 | 0.02 | 0.77 |
| S.parauberis | NC_015558.1 | 27.8 | 0.1548 | 0.04 | 6.95 |
| S.pasteurianus | NC_015600.1 | 27.2 | 0.1587 | 0.03 | 5.12 |
| S.penaeicida | NZ_LOCM00000000.1 | 24.3 | 0.1798 | 0.01 | 4.9 |
| S.periodonticum | NZ_CP034543.1 | 29.3 | 0.1461 | 0.08 | 3.55 |
| S.peroris | NZ_AEVF00000000.1 | 25.9 | 0.1675 | 0.01 | 3.35 |
| S.pharyngis | NZ_VOHL00000000.1 | 23 | 0.19 | 0 | 0.13 |
| S.phocae | NZ_LHQM00000000.1 | 26.3 | 0.1648 | 0.02 | 3.03 |
| S.plurextorum | NZ_AUI000000000.1 | 26.4 | 0.1639 | 0.02 | 1.4 |
| S.pneumoniae | NZ_LN831051.1 | 27.1 | 0.1595 | 0.03 | 2.77 |
| S.porci | NZ_AUIP00000000.1 | 23.3 | 0.1877 | 0 | 1.74 |
| S.porcinus | NZ_LS483388.1 | 25.8 | 0.1681 | 0.01 | 5.69 |
| S.pseudoporcinus | NZ_AEUY00000000.2 | 27.2 | 0.1591 | 0.03 | 5.37 |
| S.pyogenes | NZ_LN831034.1 | 26.7 | 0.1622 | 0.02 | 4 |
| S.ratti | NZ_CP043405.1 | 29 | 0.148 | 0.07 | 1.6 |
| S.respiraculi | NZ_CP022680.1 | 26.4 | 0.1643 | 0.02 | 0.52 |
| S.rubneri | NZ_SRRP00000000.1 | 28.3 | 0.1522 | 0.05 | 0.93 |
| S.ruminantium | NZ_AP018400.1 | 25.7 | 0.1688 | 0.01 | 2.49 |
| S.salivarius | NZ_LR134274.1 | 28 | 0.154 | 0.04 | 2.37 |
| S.sanguinis | NZ_LS483385.1 | 27 | 0.1604 | 0.03 | 0.83 |
| S.sinensis | NZ_JPEN00000000.1 | 26.4 | 0.1642 | 0.02 | 0.34 |
| S.sobrinus | NZ_AUUD00000000.1 | 23.9 | 0.1824 | 0 | 0.92 |
| S.suis | NZ_LS483418.1 | 26.6 | 0.1628 | 0.02 | 1.1 |
| S.thermophilus | NZ_CP038020.1 | 27.3 | 0.1579 | 0.03 | 3.48 |
| S.thoraltensis | NZ_ARCI00000000.1 | 23.5 | 0.1858 | 0 | 4.1 |
| S.troglodytae | NZ_AP014612.1 | 25.9 | 0.1679 | 0.01 | 5.32 |
| S.urinalis | NZ_LR134323.1 | 28.4 | 0.1511 | 0.05 | 8.22 |
| S.ursoris | GCA_012843165.1 | 26.6 | 0.1629 | 0.02 | 1.72 |
| S.varani | NZ_CTEN00000000.1 | 23.3 | 0.1877 | 0 | 2.09 |
| S.vestibularis | NZ_LR134275.1 | 28.2 | 0.1524 | 0.05 | 2.85 |

### 7. Bacteriostatic Activity

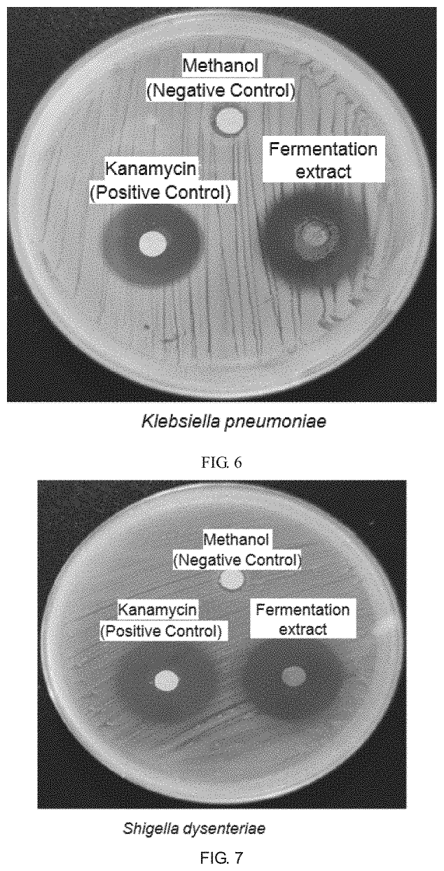

A LB fermentation medium was employed to ferment Streptococcus sp. 121 to obtain the corresponding antimicrobial active substances, and the bacteriostatic effect of the fermentation broth was determined by a filtering paper agar diffusion method. The specific operation steps were as follows. The results were shown in FIGS. 6 and 7, and the specific analysis results were shown in Table 5.

### 7.1 Preparation of fermentation extract of Streptococcus sp. 121:

10-15 single colonies of Streptococcus sp. 121 with similar morphologies were picked from the blood agar plate by an inoculating loop, and inoculated into 1500 mL of LB fermentation broth (the LB fermentation broth includes the following components in weight ratio: 10.0 g/L of tryptone; 5.0 g/L of yeast extract; 10.0 g/L of NaCl; PH 7.0), each fermentation broth was 1500 mL, and was placed and incubated in a 37° C. shaker at 150 rpm/min for 60 h. After the fermentation broth was centrifuged at 13,000 rpm/min for 15 min, the supernatant fermentation broth was taken and extracted with ethyl acetate (1:1), and the obtained extract in ethyl acetate was concentrated by rotary evaporation and weighed to obtain 130 mg of the fermentation extract. The fermentation extract was dissolved and diluted with methanol to 1 mg/10 μL, and stored at 4° C. for later use.

### 7.2 Preparation Of MH Plate For Strains To Be Tested (Klebsiella pneumoniae and Shigella dysenteriae):

5-10 Klebsiella pneumoniae and Shigella dysenteriae with similar morphologies were respectively picked from the blood agar plate by an inoculating loop, and respectively inoculated into LB liquid medium, incubated overnight, then subjected to multiple dilution with sterilized distilled water, so as to adjust the concentration of the bacterial solution to 10⁸ CFU/mL. Then, the bacterial solutions of Klebsiella pneumoniae and Shigella dysenteriae were dipped with sterile cotton swabs, the excess bacterial solutions were squeezed out on the tube wall, and then the bacterial solutions were coated on the MH plate respectively for drying.

### 7.3 Test method (filtering paper agar diffusion method):

Three sterile filtering paper sheets with a diameter of 0.6 cm were respectively taken: one was added dropwise with 50 μL of a methanol solution (as negative control), one was added dropwise with 50 μL of ampicillin with a concentration of 1 mg/mL (as positive control of the strain to be tested Klebsiella pneumoniae) or 50 μL of kanamycin with a concentration of 1 mg/mL (as positive control of the strain to be tested Shigella dysenteriae), and the other was added dropwise with 50 μL of the fermentation extract of Streptococcus sp. 121 with a concentration of 1 mg/10 μL. The three pieces of filtering paper were clamped by sterile tweezers and attached to the MH plate already coated with the bacterial solution of the strain to be tested. After completion of the aforementioned operations, the culture dish was incubated in an incubator at 37° C. for 16-20 h, then the size of each bacteriostatic ring was measured with a vernier caliper in replicate for 3 times, and the average value was taken by calculation.

TABLE 5
Bacteriostatic results of fermentation extract of Streptococcus sp. 121

| | Klebsiella | |
|---|---|---|
| | pneumoniae | Positive control (Kanamycin) |
| The first time | 25 mm | 20 mm |
| The second | 24 mm | 18 mm |
| time | | |
| The third time | 24 mm | 21 mm |
| mean | 24.3 mm | 19.7 mm |
| | Shigella | |
| | dysenteriae | Positive control (Kanamycin) |
| The first time | 24 mm | 24 mm |
| The second | 26 mm | 24 mm |
| time | | |
| The third time | 25 mm | 23 mm |
| mean | 25 mm | 23.7 mm |

It could be seen from the bacteriostatic activity analysis results in Table 5 that the Streptococcus sp. 121 strain of the present invention was incubated in LB fermentation broth, and produced relatively stronger bacteriostatic active substances, which had obvious bacteriostatic effects on both Klebsiella pneumoniae and Shigella dysenteriae. Therefore, the strain of the present invention has a potential application prospect in the aspect of antimicrobial drugs.

The specific examples of the present invention have been described in detail above, but they are only used as examples, and the present invention is not limited to the specific embodiments described above. For those skilled in the art, any equivalent modifications and substitutions to the present invention also fall within the scope of the present invention. Therefore, all equivalent changes and modifications made without departing from the spirit and scope of the present invention should be covered within the scope of the present invention.

1. A Streptococcus, characterized in that the Streptococcus is Streptococcus sp. 121 and is registered and deposited in GuangDong Microbial Culture Collection Center with the accession number of GDMCC No: 61195.

2. A formulation for antagonizing Klebsiella pneumonia, characterized in that the formulation containing the Streptococcus according to claim 1.

3. A medicament for preventing and treating a condition caused by Klebsiella pneumonia, characterized in that the medicament containing the Streptococcus according to claim 1.

4. A formulation for antagonizing Shigella dysenteriae, characterized in that the formulation containing the Streptococcus according to claim 1.

5. A medicament for preventing and treating a disease caused by Shigella dysenteriae, characterized in that the medicament containing the Streptococcus according to claim 1.

6. A fermentation broth, or a supernatant fermentation broth obtained after centrifugation of the fermentation broth, or a extract of the supernatant fermentation broth in ethyl acetate of the Streptococcus according to claim 1.

7. A formulation for antagonizing Klebsiella pneumoniae, characterized in that the formulation containing the fermentation broth, or the supernatant fermentation broth obtained after centrifugation of the fermentation broth, or the extract of the supernatant fermentation broth in ethyl acetate of the Streptococcus according to claim 6.

8. A medicament for preventing and treating a condition caused by Klebsiella pneumonia, characterized in that the formulation containing the fermentation broth, or the supernatant fermentation broth obtained after centrifugation of the fermentation broth, or the extract of the supernatant fermentation broth in ethyl acetate of the Streptococcus according to claim 6.

9. A formulation for antagonizing Shigella dysenteriae, characterized in that the formulation containing the fermentation broth, or the supernatant fermentation broth obtained after centrifugation of the fermentation broth, or the extract of the supernatant fermentation broth in ethyl acetate of the Streptococcus according to claim 6.

10. A medicament for preventing and treating a disease caused by Shigella dysenteriae, characterized in that the formulation containing the fermentation broth, or the supernatant fermentation broth obtained after centrifugation of the fermentation broth, or the extract of the supernatant fermentation broth in ethyl acetate of the Streptococcus according to claim 6.
